(19) United States
(12) Patent Application Publication
(10) Pub. No.: US 2012/0282193 A1
(43) Pub. Date: Nov. 8, 2012

(54) AGENT FOR THE PREVENTION AND TREATMENT OF THE INITIAL DENTAL CARIES

(75) Inventor: Martin MARYSKA, Praha (CZ)

(73) Assignee: GORAM SPOL. S.R.O.

(21) Appl. No.: 13/552,729

(22) Filed: Jul. 19, 2012

Related U.S. Application Data
(62) Division of application No. 12/173,894, filed on Jul. 16, 2008.
Application No. 12/173,894 is a continuation-in-part of application No. PCT/CZ2006/000091, filed on Dec. 14, 2006.

(30) Foreign Application Priority Data
Jan. 19, 2006 (CZ) PV2006-40

(51) Int. Cl. A61K 33/42; A61P 1/02

(52) U.S. Cl. 424/57

(57) ABSTRACT

A composition for the prevention and treatment of initial tooth caries, that is composed of a phosphate-calcium gel with the Ca/P mass ratio ranging from 1.2 to 1.5 and with a bond energy of the 2p phosphorus electron of 131 eV to 136 eV and optionally containing at least one auxiliary dental substance. The composition can further contain an aqueous solution of methyl cellulose or/and chlorohexidine gluconate.

### CROSS-REFERENCE TO RELATED APPLICATION

This application is a divisional of application Ser. No. 12/173,894, which is a continuation-in-part of International Application No. PCT/CZ2006/000091 filed on Dec. 14, 2006, which was published in the English language on 26 Jul. 2007 with International Publication Number WO 2007/082496 A3, and which claims priority from CZ Patent Application Number PV2006-40 filed 19 Jan. 2006, the contents of which is incorporated herein by reference.

### FIELD OF THE INVENTION

The invention deals with an agent for the prevention and treatment of the initial dental caries.

### BACKGROUND OF THE INVENTION

The dental caries represents a chronic infectious disease that progresses very slowly in the majority of individual persons. The disease may affect the enamel, the dentine or the cement and it is not characterized by any selflimiting factors which means that it may lead to a complete destruction of the tooth if it is not treated.

The tooth caries occurring on the surface of the dental enamel is a process reflecting the metabolic activity taking place in the biofilm of the dental plaque. In the course of time the balance between the mineral surface of the enamel and the surrounding dental plaque liquid is disturbed. The loss of the hard tooth tissue by demineralization results eventually in the formation of a carious lesion.

The tooth caries is a multifactorial disease with a variety of causes. Some factors are necessary for the development of the caries, in particular the presence of the dental plaque biofilm on the enamel surface, the presence of carbohydrates and the susceptible surface of the enamel; other factors are supplementary, eg the composition of the food and the frequency of its consumption, fluorides, buffer capacity of the saliva, etc.

The development and the progression of the initial decay lesion are described by some experimental studies.

The methods preventing the formation of the dental caries have made a great progress in the last half a century. These methods encompass the regular mechanical removal of the dental plaque biofilm by various techniques and antimicrobial agents, the dietary counseling aimed at decreasing the frequency of consumption of sweet food and drinks as well as the most successful preventive techniques based on the application fluoride ions into the enamel hydroxyapatite. The prevention of the dental caries by fluorides set out from the idea of the substitution of the hydroxyl groups in the enamel hydroxyapatite by fluorine ions leading to its resistance to the dissolution in acids; the formed fluorohydroxyapatite or fluorapatite makes the enamel surface extraordinarily resistant against the dental caries development.

In addition to this embedment of fluoride ions in the enamel hydroxyapatite through the systemic supply of fluorides (fluoridated drinking water, NaF tablets, fluoridated salt and fluoridated milk) an extraordinarily efficient local action of fluoride ions on the surface layer of the enamel was discovered in the 1980s. It was found that if fluoride ions are present in the saliva or in the dental plaque liquid they enter spontaneously into the surface layer of the enamel into the depth of about 20 to 30 m thus making it extremely resistant to the acid attack. At present, this local action of fluorides is regarded as the basic method of dental caries prevention.

At the time present a wide range of various dental hygiene means characterized by local fluoride action are being used with the aim to prevent the dental caries: fluoridated tooth pastes, fluoride gels to be applied at the dental chair as well as in daily use at home, mouthrinses with a higher fluorine content for mouth washing, fluoridated dental flosses and fluoridated chewing gums. Various calcium phosphates that should encourage the formation of dental enamel hydroxyapatite also occur in a variety of toothpastes. However, the minimum size of all ingredients added so far into currently used toothpastes and other fluoride containing agents are in the order of several to tens of micrometers. Such huge particles are not able to penetrate into carious microdefects in the tooth enamel and influence their repair.

A pronounced development has taken place hi the field of another extraordinary invention in the past decade—the application of the nanotechnologies. Nanomaterials with the particle size ranging from 10 to 100 nm exhibit other properties than identical materials composed of the grains having the size of hundreds of nanometers or being even larger. Among other things, also the processes suitable for the preparation of nanoparticles of hydroxyapatite and other well-defined calcium phosphates were developed. Their application in the dentistry is also mentioned in patent literature but no such material has been applied in the practice so far. Obviously, this is due to the fact that the techniques that would enable to investigate the tooth microstructure and its changes resulting from the use of therapeutic processes “in vivo” are not currently available. The electron microscopy is applied almost exclusively to the investigation into the enamel microstructure by using extracted teeth on which, however, various treatment effects can not be studied any longer.

A mixture containing nanoparticles of hydroxyapatite used for a rapid repair of enamel defects is described hi the technical literature (K. Yamagishi, K. Onuma, T. Suzuki, F. Okada, J. Tagami, M. Otsuki, P. Senawangse: A synthetic enamel for rapid tooth repair. Nature, vol. 433, 24 Feb. 2005, p. 819). Nevertheless, the workers use a mixture of HAP nanoparticles in a very acid environment, which affects unfavorably the mucous membrane in the oral cavity.

The subject of the present invention is to offer an agent that—in comparison with the known materials used up to now—would enable an even more efficient prevention and cure of the initial dental caries lesion.

### SUMMARY OF INVENTION

The above mentioned subject has been achieved by using an agent for prevention and curing of the initial tooth caries according to this invention. Thus, the subject matter to the invention is the agent for the prevention and treatment of the initial dental caries on the basis of calcium phosphate which comprises a pre-formed gel consisting of phosphate-calcium with the Ca/P mass ratio ranging from 0.9 to 1.5, preferably from 1.2 to 1.5 and with the bond energy of the 2p phosphorus electron of 131 eV to 136 eV.

The agent according to the invention is preferably prepared as described in the Examples, i.e. by a sol-gel precipitation method wherein an alkaline solution of Ca²⁻ ions is mixed with an alkaline solution of HPO₄²⁻ ions in an aqueous medium.

More specifically the solution of Ca²⁻ ions can be poured into the solution of HPO₄²⁻ ions under vigorous stirring; as the solution of Ca²⁻ ions a solution of calcium nitrate in the mixture of water and ethanol, and as the solution of HPO₄²⁻ ions a solution of ammonium monohydrogen phosphate in water are used; the pH value of both the solutions are used in amounts corresponding to Ca/P mass ratio 2.15; and the resulting gel is separated and washed.

The separation of the product can be carried out by filtration and then the product can be washed with water and ethanol.

The agent according to the invention can further contain at least one auxiliary dental substance such as an aqueous solution of methyl cellulose. Proteins of the enamel matrix (amelogenine, ameloblastine, enameline and tufteline), monetite, sodium fluoride and taste improving additives may also be used as additional additives.

This agent can be applied in form of toothpaste, paste, chewing gum, coated dental floss, foam, gel or varnish.

### DESCRIPTION OF THE PICTURES SHOWN IN FIGURES

On the attached drawings:

FIG. 1

The surface make-up of the enamel of a 14-year-old person with symptoms of an initial decay process (the porosity starting to develop) are shown in Fig. Ia and Fig. Ib. Considered from the viewpoint of current present-day diagnostic methods this would be a tooth without any sign of the clinically identifiable decay activity. Perikymata are visible as well as the contours of the enamel prisms. The pictures were taken “in vivo” with the aid of an optical microscope.

FIG. 2

This Figure shows the development of a sub-surface porosity: FIG. 2a—individual pores at the enamel surface; FIG. 2b, c, d—the pore coalescence (various types). AU pictures were obtained “in vitro” with the aid of a scanning electron microscope (SEM).

FIG. 3

FIGS. 3a through 3f show, in detail, individual stages of the development in the initial decay lesion. FIG. 3a shows the formation of pores and their coalescence, FIG. 3b shows the porosity development, the pore coalescence and micro-fissures, FIG. 3c shows the pore coalescence and micro-cavitation, FIG. 3d shows pores in the wall of a micro-fissure, FIG. 3e shows the micro-cavitation and the beginning pitting effect in the depth of a micro-cavity and FIG. 3f shows the micro-cavitation of a initial decay with a broken-off surface layer of the enamel. All pictures were taken “in vitro” with the aid of a scanning electron microscope.

FIG. 4

FIGS. 4a and 4b show the defect repair in the tooth surface under the action of a calcium fosfate gel. All pictures were taken “in vitro” with the aid of a scanning electron microscope.

FIG. 5

FIG. 5a through FIG. 5d demonstrates the reparative effect of a calcium phosphate gel “in vivo”. FIGS. 5a and 5b show the initial surface state of the first upper premolar (considered from the viewpoint of current present-day diagnostic methods this would be a tooth without any sign of the clinically identifiable decay activity—see Fig. Ia and Ib), FIGS. 5c and 5d demonstrate a reparative action of a calcium phosphate gel applied in the faun of a toothpaste for 14 days; two-minute tooth cleaning twice a day, 14-year-old patient. The pictures made “in vivo” with the aid of an optical microscope.

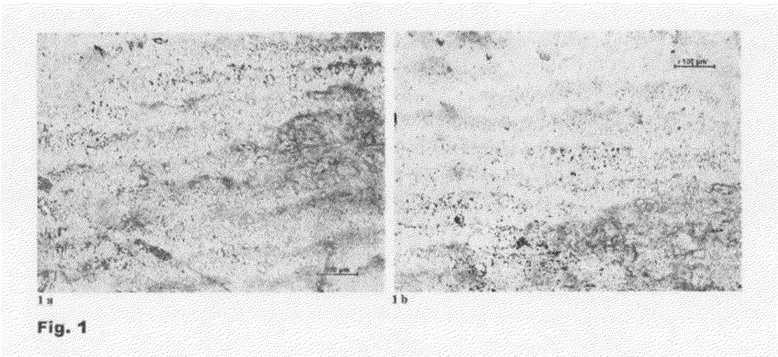

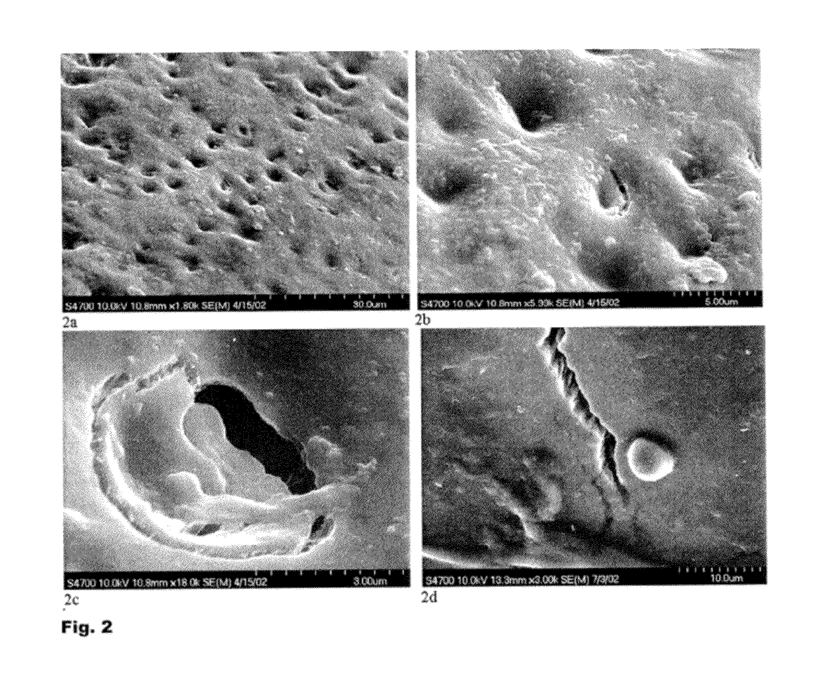

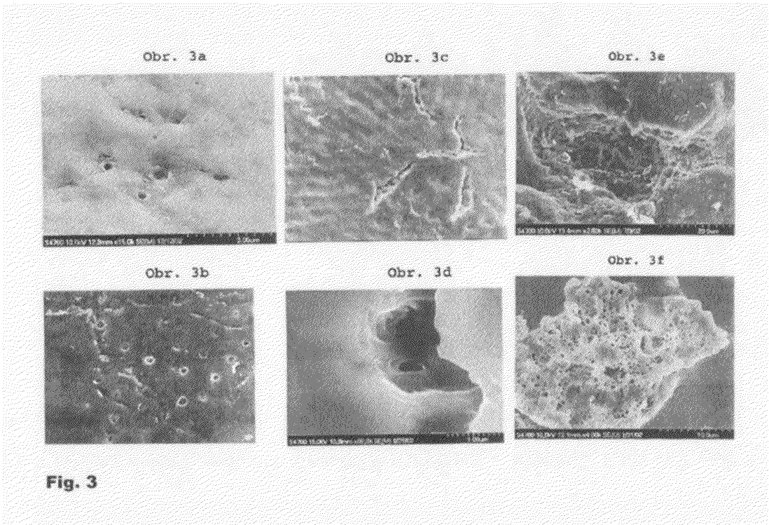

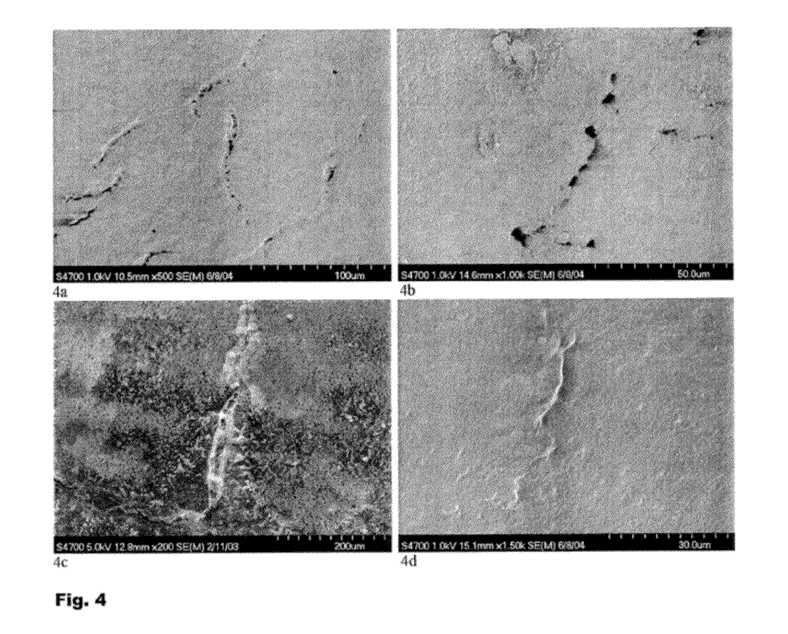

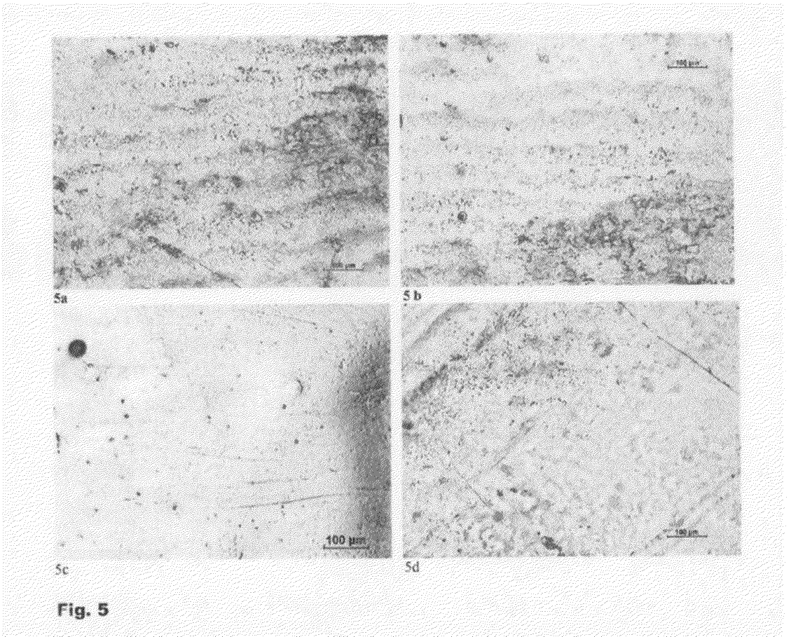

### EXAMPLES

### Preparation of the Gel with the Ca/P Mass Ratio=1.3

1. Preparation of the Ca(NO₃)₂4.H₂O Solution

116.8 g of Ca(NO₃)₂.4H₂O are dissolved at 37° C. in 50 milliliters of the mixture of distilled water with ethanol at a ratio 1:1 and the pH value of the solution obtained in such a way is adjusted with concentrated ammonia to pH=9 (approximately 0.4 milliliters).

2. Preparation of the (NH₄)₂HPO₄ Solution

39.1 g of (NH₄)₂HPO₄ are dissolved in 50 milliliters of distilled water at 37° C. and the pH value of the solution prepared in such a way is adjusted with concentrated ammonia to pH=9 (4 milliliters).

Both solutions are mixed together at a temperature of 37° C., the mixture is stirred continuously and intensely with a magnetic stirrer while the solution of calcium nitrate is poured into the solution of ammonium phosphate. The final product is filtered several times immediately after the end of the stirring lasting for 3 minutes. The product on the filter is first rinsed once with warm distilled water, then with the water/ethanol solution (volume ratio 1:1) and, finally, with ethanol only. The filtering is carried out with the aid of a water jet pump by using the S4 fit and “blue ribbon” paper filter.

The resulting product is characterized by the bond energy of the P₂ₚ phosphorus electron ranging from 135 eV to 132 eV and by the Ca/P mass ratio of about 1.3.

### Example 1

5 g of the product prepared in the above mentioned way is dispersed in the 2-percent solution of tylose and 25 milliliters of 0.1-percent chlorohexidine are added to the mixture obtained; this mixture is then homogenized by ultrasound (100 W for a period lasting 5 minutes). The final product is thin and shows a tendency towards sedimentation; therefore, it is suitable for immediate application. It may be necessary to stir it thoroughly before use.

### Example 2

5 g of the product are dispersed in the 2-percent solution of tylose (tylose dissolved in 50 milliliters of water, 25 milliliters of the 0.1-percent chlorohexidine added to the solution and made up to the mark of 100 milliliters). First, a small amount of this tylose solution is added to the weighed amount of gel (5 g to obtain a mixture containing 5%), the mixture is stirred thoroughly, then the remaining quantity of tylose is added, the mixture is stirred thoroughly once again with a glass rod and, subsequently, the homogenization by ultrasound similar to that mentioned in Example 1 takes place. The resulting product is more viscous than that mentioned in Example 1 but it still exhibits a tendency towards sedimentation and, therefore, it is also suitable for immediate use or it may be necessary to stir it thoroughly before use.

### Example 3

10 g of the product are dispersed in the 4-percent solution of tylose (tylose dissolved in 60 milliliters of water, 25 milliliters of the 0.1-percent chlorohexidine added to the solution and made up to the mark of 100 milliliters directly in the beaker). First, a small amount of this solution of tylose is added to the weighed amount of gel (5 g to obtain a mixture containing 5%), the mixture is stirred thoroughly, then the remaining quantity of tylose is added, the mixture is stirred thoroughly once again with a glass rod and, subsequently, the homogenization by ultrasound similar to that mentioned in Example 1 takes place. The resulting consistency of the product meets the requirements for current applications, ie it sediments negligibly.

### Example 4

10 g of the product are dispersed in the 4-percent solution of tylose (tylose dissolved in 60 milliliters of water, 25 milliliters of the 0.1-percent chlorohexidine added to the solution and made up to the mark of 100 milliliters directly in the beaker). First, a small amount of this solution of tylose is added to the weighed amount of gel (5 g to obtain a mixture containing 5%), the mixture is stirred thoroughly, then the remaining quantity of tylose and 5 milliliters of a strawberry syrup are added, the mixture is stirred thoroughly once again with a glass rod and, subsequently, a five-minute treatment by ultrasound takes place. The resulting consistency of the product is the same as that in Example 3.

### Example 5

The same procedure as that described in Example 4 is used except for the fact that 1 percent of a mixture of proteins of the glassy matrix (amelogenine, ameloblastine, enameline and tufteline) is added.

### INDUSTRIAL APPLICABILITY

The agent as described in this invention can be used practically in the form of rinsing mouth suspension, rinsing suspension from artificial saliva, gel compress containing a gel with the Ca/P mass ratio equal to 1.2 . . . 2.2, toothpaste containing sol, respectively gel with the Ca/P mass ratio equal to 1.2 . . . 2.2, toothpastes to be used in conjunction with a standard or ultrasonic toothbrush, chewing gums containing a gel with the Ca/P mass ratio equal to 1.2 . . . 2.2, high-viscosity mixtures containing a gel with the Ca/P mass ratio equal to 1.2 . . . 2.2 to be used a) as a fissure sealing agent, b) on the interdental toothbrush to treat the approximal surfaces of the tooth, c) on dental thread to treat approximal surfaces.

The agent according to invention is suitable for both the preventative care and curative purposes aimed at treating the initial tooth caries.

As regards the home care, toothpastes, pastes, chewing gums, dental threads and foam come into consideration. Gel, powder and paint could then be used within the framework of the professional care.

1-6. (canceled)

7. A method of the treatment of initial symptoms of tooth decay process, such as porosity of the dental enamel, which comprises applying on the teeth suspected of having such symptoms an agent comprising a calcium phosphate gel with a Ca/P mass ratio ranging from 0.9 to 1.5 and with bond energy of the 2p phosphorus electron of 131 eV to 136 eV.

8. The method according to claim 7, wherein the Ca/P mass ratio ranges from 1.2 to 1.5.

9. The method according to claim 7, wherein said gel is prepared by sol-gel precipitation method wherein an alkaline solution of Ca²⁺ ions is mixed with an alkaline aqueous solution of HPO₄²⁻ ions in an aqueous medium.

10. The method according to claim 9, wherein the solution of Ca²⁺ ions is poured into the solution of HPO₄²⁻ ions under vigorous stirring; as the solution of Ca²⁺ ions a solution of calcium nitrate in the mixture of water and ethanol, and as the solution of HPO₄²⁻ ions a solution of ammonium monohydrogen phosphate in water are used; the pH value of both the solutions is adjusted to 9 by ammonia; the solutions are used in amounts corresponding to Ca/P mass ratio 2.15; and the resulting gel is separated and washed.

11. The method according to claim 10, wherein the gel is separated by filtration then washed with water and ethanol.

12. The method according to claim 7, wherein said agent further comprises at least one auxiliary dental substance.

13. The method according to claim 7, wherein said agent is in the form of a toothpaste.
